(19) United States
(12) Patent Application Publication
(10) Pub. No.: US 2025/0228694 A1
(43) Pub. Date: Jul. 17, 2025

(54) SYSTEM FOR CONTROLLING FECAL INCONTINENCE

(71) Applicant: Christopher W. CROWLEY, Solana Beach, CA (US)

(72) Inventor: Ying-Tzy LIN, San Diego, CA (US)

(21) Appl. No.: 18/412,502

(22) Filed: Jan. 13, 2024

(51) Int. Cl. A61F 5/44; A61F 5/451

(52) U.S. Cl. CPC A61F 5/4408; A61F 5/451

(57) ABSTRACT

An apparatus for controlling the collection of fecal matter from an incontinent person includes a harness which can be worn by the person. As part of the apparatus, a combination of straps is provided which can be manipulated to open and close a potential space between a pair of the straps. The harness can be adjusted to properly position the potential space for collection of fecal matter. Also included is a fecal collection receptacle which has an opening that operates in conjunction with the manipulation of the potential space. In operation, the collection receptacle is attached to the harness and then periodically removed either separately or in combination with the harness for disposal.

### FIELD OF THE INVENTION

The present invention pertains generally to apparatuses that can be externally worn by a person to manage and facilitate patient care of uncontrolled bodily functions. More particularly, the present invention pertains to apparatuses that help a person deal with issues associated with fecal incontinence. The present invention is particularly, but not exclusively, useful as a harness that can be worn by a person to efficiently collect uncontrolled fecal matter eliminations for appropriate disposal.

### BACKGROUND OF THE INVENTION

Fecal incontinence is a devastating affliction that significantly impacts a person's quality of life. It is an affliction that is uncontrollable and may happen with little or no warning. Thus, it requires special care that must be given immediately. Under these conditions, not only does the incontinent person suffer, the lives of immediate family and friends will also be unavoidably impacted. Succinctly stated, fecal incontinence is horrible for health and social reasons.

Health and sanitary considerations dictate that each fecal incontinent episode be attended to as soon as possible, with utmost care. A substantial concern is the immediate collection as well as the ultimate disposal of fecal matter, and the care and cleaning of the individual in preparation for the next episode. Unfortunately, it happens there may be as many as three or four episodes within any 24-hour period.

It is of course very possible that, with the proper apparatus, a person suffering fecal incontinence may reasonably expect to have a relatively normal quality of life.

With the above in mind, it is an object of the present invention to provide an apparatus for the collection of fecal matter with minimal intrusion into an incontinent person's daily activities. Another object of the present invention is to give a person with fecal incontinence a reassuring degree of “peace of mind”. Still another object of the present invention is to provide an apparatus for the collection of fecal matter that is simple to manufacture, is easy to use, and comparatively cost effective.

### SUMMARY OF THE INVENTION

An apparatus for controlling the collection of fecal matter in accordance with the present invention comprises a harness which can be worn by a person suffering with fecal incontinence. Within the harness itself is a pair of opposed edge straps which are positioned in a side-by-side relationship with each other. The two edge straps are fixedly joined together by an upper grip and a lower grip, which are located with a separation distance established between the grips. Operationally, when opposed lateral forces are exerted outwardly on respective edge straps in a direction away from the separation distance, the side-by-side relationship between the edge straps is ended. They then spread apart or are returned, to create a potential space there between.

The designation of a potential space is commonly used to describe specific anatomical structures in which tissues are arranged in apposition and may be opened or closed. For the present invention, the potential space is understood in a broader sense. Applying to both tissues so-arranged as well as adjacent external structures such as the edge straps that may be spread apart or returned to the closed side-by-side arrangement.

To assist in closing an opened potential space, a cinch may be provided to interconnect the opposed edge straps. When the lateral forces that created the open potential space are released, the cinch causes the edge straps to resume their side-by-side relationship with each other, to thereby return the potential space to its closed configuration. In a preferred embodiment, the cinch includes a locking mechanism to secure the edge straps together to facilitate sanitary removal and disposal of fecal matter.

The purpose of other straps in the harness are to establish and maintain the location of the potential space where it can be effectively used for its intended purpose, i.e. facilitate the collection of fecal matter. Both the edge straps as well as other straps in the harness may be stretchable or non-stretchable and may have circular, rectangular or square cross sections. In a preferred embodiment, the edge straps are made from an elastic material.

Also included with the apparatus of the present invention is a fecal collection or recovery receptacle. This receptacle is collapsible, and it has an opening that is surrounded by a flexible periphery. Dimensionally, this opening is sized to establish a fluid tight engagement with the edge straps and the grips which surround the potential space between the upper grip and the lower grip. Operationally, during this engagement, the flexible periphery of the collapsible receptacle remains engaged with the edge straps of the harness as the potential space is transformed between its closed and its opened configurations. For its use, the collapsible receptacle will preferably be engaged with the potential space when the potential space is in its closed configuration. The potential space can then be opened while it is being worn. After use, the potential space can be closed and either the entire harness of only the receptacle can be removed

As envisioned for the present invention, the harness will include a waist belt which is interconnected by other straps with the edge straps and the grips that surround the potential space. For a preferred embodiment of the present invention, these other straps include a back strap, a pair of leg straps, and a pair of adjustable lateral straps.

In detail, the back strap interconnects the upper grip with the waist belt. The pair of leg straps interconnect the lower grip with the waist belt as they pass through the legs and straddle the person's genitalia. As for the adjustable lateral straps, a left lateral strap interconnects the left edge of the potential space with the waist belt and a right lateral strap interconnects the right edge of the potential space with the waist belt. In this combination, adjustments of the lateral straps are alternately tensioned to open the potential space and receive fecal matter into the collection receptacle, or they are relaxed to close the potential space and thereby collapse the potential space for removal of the entire harness assembly or the collection receptacle.

Another feature of the present invention is a protective covering which is attached to the flexible periphery around the opening of the collapsible receptacle. Specifically, the protective covering must be adaptable to expand from a compressed condition to cover the potential space when the potential space is in an open configuration. As envisioned for the present invention the protective covering will preferably be a layer selected from the group consisting of a paper fiber, cloth fiber or a film which is formed with a hole to allow fluids and semi-fluids to flow there through. As an alternative or addition to the protective covering a barrier can be employed which may be ointment, a gel, an oil, a cream, a topical medicament, a wax or a petroleum derivative applied to the person under the potential space. Further, a dispenser can be attached to the edge straps to administer barrier material as the potential space is moved into an open configuration. Further, the cross section and material properties of the edge straps may be configured to sweep the protective covering materials together with remnant fecal matter into the receptacle as the potential space is moved to the close position.

### DESCRIPTION OF THE DRAWINGS

The novel features of this invention, as well as the invention itself, both as to its structure and operation, will be best understood from the accompanying drawings, taken in conjunction with the accompanying description, in which similar reference characters refer to similar parts and in which:

FIG. 1 is a backside view of an assembled apparatus shown positioned for wear on the rear end of a manikin;

FIG. 2A is a perspective view of a collapsible receptacle with the flexible periphery of its opening in a closed configuration;

FIG. 2B is a perspective view of the collapsible receptacle shown in FIG. 2A with the flexible periphery of its opening in an opened configuration;

FIG. 3A is a perspective view of a potential space in accordance with the present position shown in a closed configuration;

FIG. 3B is a perspective view of the potential space shown in FIG. 3A in an opened configuration; and

FIG. 3C is a perspective view of the potential space shown in FIG. 3B with a protective covering extending across the potential space.

### DESCRIPTION OF THE PREFERRED EMBODIMENTS

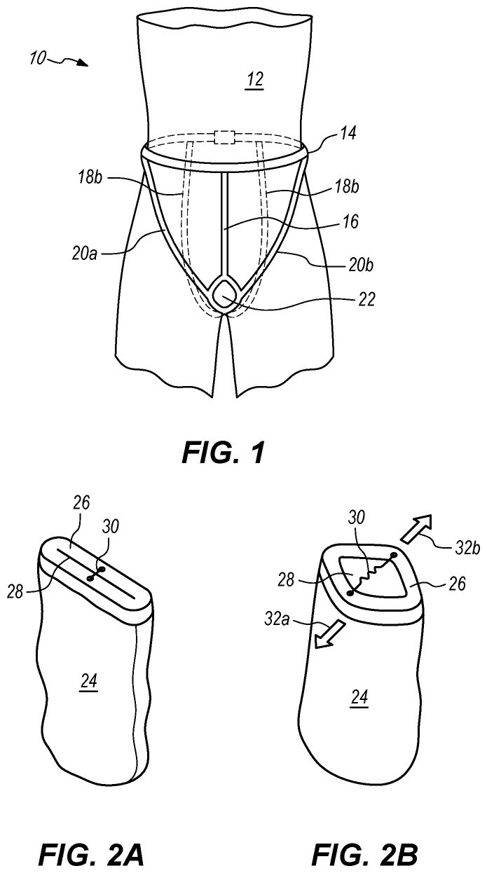

Referring initially to FIG. 1, an apparatus for controlling the collection of fecal matter in accordance with the present invention is shown and is generally designated 10. As shown the apparatus 10 is positioned on a manikin 12 to show the apparatus 10 from a backside perspective as a combination of different straps.

In detail, apparatus 10 includes a waist belt 14 to which several other straps are connected. In combination, the connecting straps include a back strap 16, a left leg strap 18a which is shown in phantom against the front portion of the manikin 12, and a right leg strap 18b also shown in phantom against the portion of the manikin 12. Other connecting straps include a left lateral strap 20a and a right lateral strap 20b, both of which are shown primarily positioned against the back side of the manikin 12. FIG. 1 shows the back strap 16 and the left and right leg straps 18a and 18b are fixed. On the other hand, the left and right lateral straps 20a and 20b are adjustable and are shown respectively interconnecting with the waist belt 14 for the purpose of manipulating the potential space 22.

FIG. 2A shows a collapsible receptacle 24 which has a flexible periphery 26 that surrounds an opening 28. As shown in FIG. 2A, the opening 28 is shown being held closed by a cinch 30. Specifically, the cinch 30 imposes forces on the flexible periphery 26 that are biased to hold the flexible periphery 26 in its closed configuration. FIG. 2B shows the collapsible receptacle 24 with counter forces 32a and 32b acting on the flexible periphery 26 to thereby establish the opening 28 in the open configuration shown in FIG. 2b. Operationally, the counter forces 32a and 32b which open the collapsible receptacle 24 are respectively applied by pulling actions on the left lateral strap 20a and the right lateral strap 20b. Also as shown in FIG. 2B, the cinch 30 is positioned adjacent to the flexible periphery 26 to avoid blocking the opening 28.

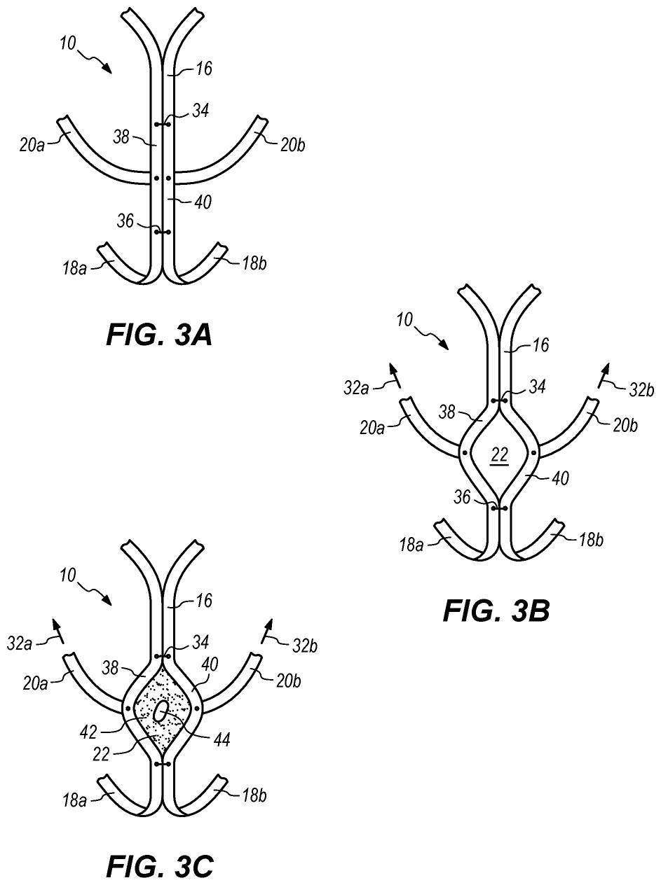

Referring now to FIG. 3A, apparatus 10 is shown to include both an upper grip 34 and a lower grip 36. The grips 34 and 36 are employed to hold a left edge strap 38 against a right edge strap 40 at respective locations which are separated from each other by a predetermined separation distance. Approximately midway between the upper grip 34 and the lower grip 36, the left lateral strap 20a is connected to the left edge strap 38, and the right lateral strap 20b is connected to the right edge strap 40.

As shown in FIG. 3B. when a counter force 32a is applied to the left lateral strap 20a, and a counter force 32b is applied to the right lateral strap 20b, the left edge strap 38 is separated from the right edge strap 40 and the potential space 22 is thereby established. FIG. 3C further shows that a protective covering 42 can be pre-connected with the left edge strap 38 and the right edge strap 40 to cover the potential space 22. Also, the protective covering 42 can be formed with a hole 44 to allow liquid and semi-liquid fluids to pass there through during a fecal incontinence episode.

Preferably, for an operation of the apparatus 10, the flexible periphery 26 of the collapsible receptacle 24 is attached to the left edge strap 38 and to the right edge strap 40 when the collapsible receptacle 24 is configured as shown in FIG. 2A, and the potential space 22 is closed as shown in FIG. 3A. Furthermore, it is preferable for the collapsible receptacle 24 to be disconnected from apparatus 10 for disposal when the collapsible receptacle 24 is configured as shown in FIG. 2A, and the potential space 22 is closed as shown in FIG. 3A. In an alternative embodiment, the apparatus 10 may be removed for disposal.

While the particular invention as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. An apparatus for controlling the collection of fecal matter which comprises:
an elongated central strap having a first end and a second end with a slit extending lengthwise there between to create a left half and a right half for the central strap;
an upper grip for holding the left half of the central strap in constant contact with the right half of the central strap at a first location on the central strap;
a lower grip for holding the left half of the central strap in constant contact with the right half of the central strip at a second location on the central strap to establish a separation distance between the upper grip and the lower grip and to create a potential space there between along the separation distance, and wherein the potential space is bounded by a left edge and a right edge between the upper grip and the lower grip;
a collapsible collection receptacle having an opening surrounded by a flexible periphery dimensioned for a fluid tight engagement with the central strap around the potential space between the upper grip and the lower grip;
a waist belt;
a back strap segment of the central strap for interconnecting the upper grip with the waist belt;
a pair of leg straps for interconnecting the lower grip with the waist belt; and
a pair of adjustable lateral straps, with a left lateral strap interconnecting the left edge of the potential space with the waist belt and with a right lateral strap interconnecting the right edge of the potential space with the waist belt, wherein adjustments of the lateral straps are alternately tensioned to open the potential space and receive fecal matter into the collection receptacle and relaxed to close the potential space and thereby collapse the potential space for removal of fecal matter.

2. An apparatus as claimed in claim 1 further comprising a protective covering adaptable to cover the potential space when the potential space is in an open configuration.

3. The apparatus of claim 2 wherein the protective covering is a layer selected from the group consisting of an ointment, a gel, an oil, a cream and a topical medicament.

4. The apparatus of claim 2 wherein the protective covering is a barrier selected from the group consisting of a wax and a petroleum derivation.

5. The apparatus of claim 2 wherein the protective covering is a cover selected from the group consisting of a paper fiber, a cloth fiber and a film.

6. The apparatus of claim 5 wherein the protective covering is formed with a hole.

7. The apparatus of claim 1 further comprising a cinch interconnecting the left edge with the right edge of the potential space at a point intermediate the separation distance between the upper grip and the lower grip to bias the lateral straps to collapse the potential space.

8. The apparatus of claim 7 further comprising a locking mechanism to hold the collapsed potential space in a closed position for sanitary disposal.

9. An apparatus for controlling the collection of fecal matter which comprises:
a collapsible receptacle for collecting fecal matter, wherein the collapsible receptacle is formed with an opening having a flexible periphery;
a harness, wherein the harness includes an arrangement of straps operable to create a potential space, wherein the potential space is reconfigurable between an open configuration and a closed configuration, and wherein the harness is engaged with the flexible periphery of the collapsible receptacle to establish a fluid tight connection therewith around the potential space; and
a means configured within the harness for manipulating the potential space to open the potential space for receiving fecal matter into the collection receptacle, and to close the potential space for removing the collection receptacle from the harness.

10. The apparatus of claim 9 wherein the manipulating means further comprises:
an elongated central strap having a first end and a second end with a slit extending lengthwise there between to create a left half and a right half for the central strap;
an upper grip for holding the left half of the central strap in constant contact with the right half of the central strap at a first location on the central strap; and
a lower grip for holding the left half of the central strap in constant contact with the right half of the central strip at a second location on the central strap to establish a separation distance between the upper grip and the lower grip and to create a potential space there between along the separation distance, and wherein the potential space is bounded by a left edge and a right edge between the upper grip and the lower grip.

11. The apparatus of claim 10 further comprising:
a waist belt;
a back strap segment of the central strap for interconnecting the upper grip with the waist belt;
a pair of leg straps for interconnecting the lower grip with the waist belt; and
a pair of adjustable lateral straps, with a left lateral strap interconnecting the left edge of the potential space with the waist belt and with a right lateral strap interconnecting the right edge of the potential space with the waist belt, wherein adjustments of the lateral straps are alternately tensioned to open the potential space and receive fecal matter into the collection receptacle and relaxed to close the potential space and thereby collapse the potential space for removal of the collection receptacle.

12. The apparatus of claim 9 further comprising a protective covering adaptable to cover the potential space when the potential space is in an open configuration.

13. The apparatus of claim 12 wherein the protective covering is a layer selected from the group consisting of an ointment, a gel, an oil, a cream, and a topical medicament.

14. The apparatus of claim 12 wherein the protective covering is a barrier selected from the group consisting of a wax and a petroleum derivative.

15. The apparatus of claim 12 wherein the protective covering is a cover selected from the group consisting of a paper fiber, cloth fiber and a film.

16. A method for constructing an apparatus for controlling the collection of fecal matter which comprises the steps of:
providing a collapsible receptacle having an opening surrounded by a flexible perimeter;
creating a harness, wherein the harness includes an arrangement of straps operable to create a potential space, and wherein the potential space is reconfigurable between an open configuration and a closed configuration; and
engaging the flexible periphery of the collapsible receptacle with the harness to establish a fluid tight connection therewith around the potential space.

17. The method of claim 16 further comprising the step of mounting a disperser on the flexible perimeter of the collapsible receptacle to secrete a liquid substance therefrom as the potential space is reconfigured from the closed configuration to the open configuration.

18. The method of claim 17 wherein the liquid substance is selected from the group consisting of an ointment, a gel, an oil, a cream, a topical medicament, a wax and a petroleum derivative.

19. The method of claim 16 further comprising the steps of:
splitting an elongated central strap of the harness lengthwise to create a left half and a right half;
engaging an upper grip with the central strap for holding the left half of the central strap in constant contact with the right half of the central strap at a first location;
engaging a lower grip with the central strap for holding the left half of the central strap in constant contact with the right half of the central strap at a separation distance from the upper grip, with the potential space between the upper grip and the lower grip; and
employing a cinch to interconnect the right half of the central strap with the left half of the central strap at a point in the potential space intermediate the upper grip and the lower grip to bias the potential space toward the closed configuration.

20. The method of claim 19 further comprising the steps of:
affixing a left lateral strap to the left half of the central strap in the separation distance; and
affixing a right lateral strap to the right half of the central strap in the separation distance to jointly counter the bias of the cinch and thereby create the open configuration of the potential space with respective applications of opposing forces on the left and right lateral straps.

21. The method of claim 20 further comprising the steps of:
providing a waist belt;
interconnecting a back strap between the upper grip of the central strap with the waist belt; and
interconnecting a pair of leg straps between the lower grip of the central strap and the waist belt.
